(19) United States
(12) Patent Application Publication
(10) Pub. No.: US 2022/0323340 A1
(43) Pub. Date: Oct. 13, 2022

(54) HAIR RESTORATION PREPARATION

(71) Applicant: Ivelisse Melendez, Bronx, NY (US)

(72) Inventor: Ivelisse Melendez, Bronx, NY (US)

(21) Appl. No.: 17/228,277

(22) Filed: Apr. 12, 2021

(51) Int. Cl. A61K 8/9789; A61K 8/97; A61K 8/92; A61K 8/49; A61Q 5/12

(52) U.S. Cl. CPC A61K 8/9789; A61K 8/97; A61K 8/922; A61K 8/498; A61Q 5/12

(57) ABSTRACT

A natural hair product for improving and restoring the condition of hair, and promoting the strength and growth of the hair, includes filtered water, burdock powder, stinging nettle powder, peppermint, horsetail powder, lavender flowers, and hibiscus flowers, in a base of olive oil, avocado oil, vitamin E, and lavender oil. If desired, the hair preparation may also include rosemary and fenugreek powder. The method of making the hair product is also disclosed.

### FIELD OF THE INVENTION

The present invention relates to a natural hair product for improving and restoring the condition of hair, having a unique combination of organic ingredients that act on the hair so as to disentangle it, smooth the hair surface, improve the feel as well as the ability of the hair to be combed, and to promote healthy hair growth. The preparation in accordance with the present invention is an entirely natural product made from herbs, flowers, roots, natural and essential oils, which promote the strength and growth of the hair, transforming it in a subtle and harmonious way and may be used on all types of hair textures.

### BACKGROUND OF THE INVENTION

Hair is often damaged by frequent bleaching, permanent waving and dyeing and frequent washing with degreasing surfactants. Under these conditions, hair can become brittle and it lose its luster. Moreover, hair can be charged with static electricity during combing, especially during dry winter months, further damaging and knotting of the hair, making combing much more difficult. These conditions can also result in the thinning of the hair.

### SUMMARY OF THE INVENTION

In accordance with a first aspect of the invention, the hair preparation of the present invention comprises filtered water, burdock powder, stinging nettle powder, peppermint, horsetail powder, lavender flowers, and hibiscus flowers, in a base of olive oil, avocado oil, vitamin E, and lavender oil. If desired, the hair preparation may also include rosemary and fenugreek powder.

Preferably, the foregoing ingredients may be used in the following approximate proportions: 6 liters of filtered water, 1 tablespoon of burdock root powder, 3 tablespoons stinging nettle powder, 3 tablespoons peppermint, 4 tablespoons horsetail powder, 3 tablespoons lavender flowers, 3 tablespoons hibiscus flowers, 1 oz olive oil, 1 oz avocado oil, 5 drops vitamin E, and 18 drops lavender oil.

In accordance with a second aspect of the invention, a method for producing a hair preparation comprises combining filtered water, burdock powder, stinging nettle powder, peppermint, horsetail powder, lavender flowers, and hibiscus flowers, to produce a first solution; boiling the first solution to produce a second solution; allowing solids in the second solution to macerate to produce a third solution; and adding olive oil, avocado oil, vitamin E, and lavender oil to the third solution to thereby produce the hair preparation. If desired, the method may also include the step of adding rosemary and fenugreek powder to produce the first solution.

### DETAILED DESCRIPTION

The natural hair product in accordance with the present invention improves and restores the condition of hair based on a unique combination of organic ingredients that act on the hair so as to disentangle it, smooth the hair surface, improve the feel as well as the ability of the hair to be combed and to promote healthy hair growth. The is entirely natural, made from herbs, flowers, roots, natural and essential oils, which promote the strength and growth of the hair, transforming it in a subtle and harmonious way and may be used on all types of hair textures.

In accordance with a preferred embodiment, the product is comprised of the following ingredients in the following approximate proportions:
6 liters of filtered water
1 tablespoon of burdock root powder
3 tablespoons stinging nettle powder
3 tablespoons peppermint
4 tablespoons horsetail powder
3 tablespoons lavender flowers
3 tablespoons hibiscus flowers.

In addition, the following additional ingredients can be added if desired:
4 tablespoons of rosemary
¼ tablespoon fenugreek powder.

The mixture formed of the foregoing ingredients is boiled for approximately 15 minutes and the solids are then allowed to macerate in solution for approximately three days, after which the following ingredients are added and mixed thoroughly:
1 oz olive oil
1 oz avocado oil
5 drops vitamin E
18 drops lavender oil.

At this point, the preparation is ready for use. The product should be shaken well before using, and applied to the scalp twice a day with a gentle circular massage using the fingertips for 5 minutes or more. Importantly, the application of the hair preparation with massage promotes blood circulation and is a stimulus for hair restoration and growth.

The foregoing preferred embodiments described herein is set forth as exemplary, and it will be appreciated that various modifications may be made without departing from the scope and spirit of the invention which is defined by the following claims.

What I claim is:

1. A hair preparation comprising:
a. filtered water,
b. burdock powder,
c. stinging nettle powder,
d. peppermint,
e. horsetail powder,
f. lavender flowers, and
g. hibiscus flowers.

2. The hair preparation of claim 1, further comprising olive oil, avocado oil, vitamin E, and lavender oil.

3. The hair preparation of claim 2, further comprising rosemary and fenugreek powder.

4. The hair preparation of claim 1 wherein the ingredients are in the following approximate proportions:
6 liters of filtered water
1 tablespoon of burdock root powder
3 tablespoons of stinging nettle powder
3 tablespoons peppermint
4 tablespoons of horsetail powder
3 tablespoons of lavender flowers
3 tablespoons hibiscus flowers

5. The hair preparation of claim 2 wherein the ingredients are in the following approximate proportions:
6 liters of filtered water
1 tablespoon of burdock root powder
3 tablespoons of stinging nettle powder
3 tablespoons peppermint
4 tablespoons of horsetail powder
3 tablespoons of lavender flowers
3 tablespoons hibiscus flowers
1 oz olive oil
1 oz avocado oil
5 drops vitamin E
18 drops lavender oil.

6. A method for producing a hair preparation comprising:
a. combining filtered water, burdock powder, stinging nettle powder, peppermint, horsetail powder, lavender flowers, and hibiscus flowers, to produce a first solution;
b. boiling said first solution to produce a second solution;
c. allowing solids in said second solution to macerate, to produce a third solution; and
d. adding olive oil, avocado oil, vitamin E, and lavender oil.to said third solution to thereby produce said hair preparation.

7. The method of claim 6 further comprising the step of adding rosemary and fenugreek powder to produce said first solution.
